SEQUENCE LISTING (4 sequences)

SEQ ID NO: 1 - DNA, 30 bp, Escherichia coli
catgccatggtgaaagacgacgggtatgac

SEQ ID NO: 2 - DNA, 30 bp, Escherichia coli
acgcgtcgacggatgttccggcaaacgaaa

SEQ ID NO: 3 - DNA, 24 bp, Escherichia coli
gattgagctctccggcgagagttc

SEQ ID NO: 4 - DNA, 20 bp, Escherichia coli
accgggtcgttctatccgtc

(19) United States
(12) Patent Application Publication
(10) Pub. No.: US 2005/0095688 A1
(43) Pub. Date: May 5, 2005

(54) Process for the preparation of L-amino acids using strains of the family Enterobacteriaceae

(76) Inventors: Mechthild Rieping, Bielefeld (DE); Thomas Hermann, Bielefeld (DE); Mike Farwick, Essen (DE)

Correspondence Address: FITCH, EVEN, TABIN & FLANNERY, P. O. BOX 65973, WASHINGTON, DC, 20035, US

(21) Appl. No.: 10/937,598

(22) Filed: Sep. 10, 2004

Related U.S. Application Data
(63) Continuation of application No. PCT/EP03/01992, filed on Feb. 27, 2003.
(60) Provisional application No. 60/365,837, filed on Mar. 21, 2002.

(30) Foreign Application Priority Data
Mar. 13, 2002 (DE) 102 10 962.1

(51) Int. Cl. C12P 13/04; C12P 13/08; C12N 15/74; C12N 1/21

(52) U.S. Cl. 435/106; 435/252.33; 435/488

(57) ABSTRACT

The invention relates to a process for the preparation of L-amino acids, especially L-threonine, in which the following steps are carried out: a) fermentation of microorganisms of the family Enterobacteriaceae which produce the desired L-amino acid and in which at least one or more genes selected from the group comprising lpd, aceE and aceF, or nucleotide sequences coding therefor, is (are) enhanced and, in particular, overexpressed, b) enrichment of the desired L-amino acid in the medium or in the cells of the bacteria, and c) isolation of the desired L-amino acid.

### CROSS REFERENCE TO RELATED APPLICATIONS

The present application is a continuation of international application PCT/EP03/01992, which has an international filing date of Feb. 27, 2003, and which was published in English under PCT Article 21(2) on Sep. 18, 2003. The international application claims priority to German application 102 10 962.1, filed on Mar. 13, 2002, and to U.S. provisional application 60/365,837, filed on Mar. 21, 2002.

### FIELD OF THE INVENTION

The present invention relates to a process for the preparation of L-amino acids, especially L-threonine, using strains of the family Enterobacteriaceae in which at least one or more genes selected from the group comprising lpd, aceE and aceF is (are) enhanced.

### STATE OF THE ART

L-Amino acids, especially L-threonine, are used in human medicine and in the pharmaceutical industry, in the food industry and very particularly in animal nutrition.

It is known to prepare L-amino acids by the fermentation of strains of Enterobacteriaceae, especially Escherichia coli (E. coli) and Serratia marcescens. Because of their great importance, attempts are constantly being made to improve the preparative processes. Improvements to the processes may relate to measures involving the fermentation technology, e.g. stirring and oxygen supply, or the composition of the nutrient media, e.g. the sugar concentration during fermentation, or the work-up to the product form, e.g. by ion exchange chromatography, or the intrinsic productivity characteristics of the microorganism itself.

The productivity characteristics of these microorganisms are improved by using methods of mutagenesis, selection and mutant choice to give strains which are resistant to antimetabolites, e.g. the threonine analog α-amino-β-hydroxyvaleric acid (AHV), or auxotrophic for metabolites of regulatory significance, and produce L-amino acids, e.g. L-threonine.

Methods of recombinant DNA technology have also been used for some years to improve L-amino acid-producing strains of the family Enterobacteriaceae by amplifying individual amino acid biosynthesis genes and studying the effect on production.

### OBJECT OF THE INVENTION

The object which the inventors set themselves was to provide novel procedures for improving the preparation of L-amino acids, especially L-threonine.

### SUMMARY OF THE INVENTION

The invention provides a process for the preparation of L-amino acids, especially L-threonine, using microorganisms of the family Enterobacteriaceae which, in particular, already produce L-amino acids and in which at least one or more of the nucleotide sequences coding for the genes lpd, aceE and aceF is (are) enhanced.

### DETAILED DESCRIPTION OF THE INVENTION

The term “L-amino acids” or “amino acids” mentioned hereafter is to be understood as meaning one or more amino acids, including their salts, selected from the group comprising L-asparagine, L-threonine, L-serine, L-glutamate, L-glycine, L-alanine, L-cysteine, L-valine, L-ethionine, L-isoleucine, L-leucine, L-tyrosine, L-phenylalanine, L-histidine, L-lysine, L-tryptophan and L-arginine. L-Threonine is particularly preferred.

In this context the term “enhancement” describes the increase, in a microorganism, of the intracellular activity of one or more enzymes or proteins coded for by the appropriate DNA, for example by increasing the copy number of the gene or genes, using a strong promoter or a gene or allele coding for an appropriate enzyme or protein with a high activity, and optionally combining these measures.

Through the measures of enhancement, especially over-expression, the activity or concentration of the appropriate protein is generally increased at least by 10%, 25%, 50%, 75%, 100%, 150%, 200%, 300%, 400% or 500%, and at most by up to 1000% or 2000%, based on that of the wild-type protein or the activity or concentration of the protein in the starting microorganism.

The process is characterized in that the following steps are carried out:
a) fermentation of microorganisms of the family Enterobacteriaceae which produce the desired L-amino acid and in which one or more genes selected from the group comprising lpd, aceE and aceF, or nucleotide sequences coding therefor, is (are) enhanced and, in particular, overexpressed,
b) enrichment of the desired L-amino acid in the medium or in the cells of the microorganisms, and
c) isolation of the desired L-amino acid, where constituents of the fermentation broth, and/or all or part (≧0 to 100%) of the biomass, optionally remain in the product.

The microorganisms provided by the present invention can produce L-amino acids from glucose, sucrose, lactose, fructose, maltose, molasses, optionally starch or optionally cellulose, or from glycerol and ethanol. Said microorganisms are representatives of the family Enterobacteriaceae selected from the genera Escherichia, Erwinia, Providencia and Serratia. The genera Escherichia and Serratia are preferred. The species Escherichia coli and Serratia marcescens may be mentioned in particular among the genera Escherichia and Serratia respectively.

Examples of suitable strains, particularly L-threonine-producing strains, of the genus Escherichia, and especially of the species Escherichia coli, are:
Escherichia coli TF427
Escherichia coli H4578
Escherichia coli KY10935
Escherichia coli VNIIgenetika MG442
Escherichia coli VNIIgenetika M1
Escherichia coli VNIIgenetika 472T23
Escherichia coli BKIIM B-3996
Escherichia coli kat 13
Escherichia coli KCCM-10132

Examples of suitable L-threonine-producing strains of the genus Serratia, and especially of the species Serratia marcescens, are:
Serratia marcescens HNr21
Serratia marcescens TLr156
Serratia marcescens T2000.

L-Threonine-producing strains of the family Enterobacteriaceae preferably possess, inter alia, one or more genetic or phenotypic characteristics selected from the group comprising resistance to α-amino-β-hydroxyvaleric acid, resistance to thialysine, resistance to ethionine, resistance to α-methylserine, resistance to diaminosuccinic acid, resistance to α-aminobutyric acid, resistance to borrelidine, resistance to rifampicin, resistance to valine analogs such as valine hydroxamate, resistance to purine analogs such as 6-dimethylaminopurine, need for L-methionine, optionally partial and compensable need for L-isoleucine, need for meso-diaminopimelic acid, auxotrophy in respect of threonine-containing dipeptides, resistance to L-threonine, resistance to L-homoserine, resistance to L-lysine, resistance to L-methionine, resistance to L-glutamic acid, resistance to L-aspartate, resistance to L-leucine, resistance to L-phenylalanine, resistance to L-serine, resistance to L-cysteine, resistance to L-valine, sensitivity to fluoropyruvate, defective threonine dehydrogenase, optionally capability for sucrose utilization, enhancement of the threonine operon, enhancement of homoserine dehydrogenase I-aspartate kinase I, preferably of the feedback-resistant form, enhancement of homoserine kinase, enhancement of threonine synthase, enhancement of aspartate kinase, optionally of the feedback-resistant form, enhancement of aspartate semialdehyde dehydrogenase, enhancement of phosphoenolpyruvate carboxylase, optionally of the feedback-resistant form, enhancement of phosphoenolpyruvate synthase, enhancement of transhydrogenase, enhancement of the RhtB gene product, enhancement of the RhtC gene product, enhancement of the YfiK gene product, enhancement of a pyruvate carboxylase and attenuation of acetic acid formation.

It has been found that the production of L-amino acids, especially L-threonine, by microorganisms of the family Enterobacteriaceae is improved after enhancement and, in particular, over-expression of at least one or more genes selected from the group comprising lpd, aceE and aceF.

The nucleotide sequences of the genes of Escherichia coli belong to the state of the art (cf. literature references below) and can also be taken from the genome sequence of Escherichia coli published by Blattner et al. (Science 277, 1453-1462 (1997)).

The lpd, aceE and aceF genes are described inter alia by the following data:

lpd Gene

Name: dihydrolipoamide dehydrogenase (NADH-dependent), component of 2-oxodehydrogenase and E3 component of the pyruvate dehydrogenase complex, L-protein of the glycine scission complex
EC no.: 1.8.1.4
Reference: Stephens et al.; European Journal of Biochemistry 135(3), 519-527 (1983) Steiert et. al.; Journal of Bacteriology 172, 6142-6144 (1990)
Accession no.: AE000121
Alternative names: dldh, lpda
   aceE Gene
Name: pyruvate dehydrogenase, E1 component of the pyruvate dehydrogenase complex, decarboxylase component
EC no.: 1.2.4.1
Reference: Stephens et al.; European Journal of Biochemistry 133(1), 155-162 (1983) Guest et al.; Annals of the New York Academy of Sciences 573, 76-99 (1989)
Accession no.: AE000120
   aceF Gene
Name: dihydrolipoamide acetyltransferase, E2 component of the pyruvate dehydrogenase complex
EC no.: 2.3.1.12
Reference: Stephens et al.; European Journal of Biochemistry 133(3), 481-489 (1983) Guest et al.; Annals of the New York Academy of Sciences 573, 76-99 (1989)
Accession no.: AE000120

The nucleic acid sequences can be taken from the data banks of the National Center for Biotechnology Information (NCBI) of the National Library of Medicine (Bethesda, Md., USA), the nucleotide sequence data bank of the European Molecular Biologies Laboratories (EMBL, Heidelberg, Germany, or Cambridge, UK) or the DNA Databank of Japan (DDBJ, Mishima, Japan).

The genes described in the literature references cited can be used according to the invention. It is also possible to use alleles of the genes which result from the degeneracy of the genetic code or from neutral sense mutations. The use of endogenous genes is preferred.

The term “endogenous genes” or “endogenous nucleotide sequences” is to be understood as meaning the genes or alleles, or nucleotide sequences, present in the population of a species.

Enhancement can be achieved for example by increasing the expression of the genes or enhancing the catalytic properties of the proteins. Both measures may optionally be combined.

Over-expression can be achieved by increasing the copy number of the appropriate genes or mutating the promoter and regulatory region or the ribosome binding site located upstream from the structural gene. Expression cassettes incorporated upstream from the structural gene work in the same way. Inducible promoters additionally make it possible to increase expression in the course of L-threonine production by fermentation. Measures for prolonging the life of the mRNA also improve expression. Furthermore, the enzyme activity is also enhanced by preventing the degradation of the enzyme protein. The genes or gene constructs can either be located in plasmids of variable copy number or be integrated and amplified in the chromosome. Alternatively, it is also possible to achieve over-expression of the genes in question by changing the composition of the media and the culture technique.

Those skilled in the art will find relevant instructions inter alia in Chang and Cohen (Journal of Bacteriology 134, 1141-1156 (1978)), Hartley and Gregori (Gene 13, 347-353 (1981)), Amann and Brosius (Gene 40, 183-190 (1985)), de Broer et al. (Proceedings of the National Academy of Sciences of the United States of America 80, 21-25 (1983)), Lavallie et al. (BIO/TECHNOLOGY 11, 187-193 (1993)), PCT/US97/13359, Llosa et al. (Plasmid 26, 222-224 (1991)), Quandt and Klipp (Gene 80, 161-169 (1989)), Hamilton et al. (Journal of Bacteriology 171, 4617-4622 (1989)), Jensen and Hammer (Biotechnology and Bioengineering 58, 191-195 (1998)) and well-known textbooks on genetics and molecular biology.

Plasmid vectors replicable in Enterobacteriaceae, e.g. cloning vectors derived from pACYC184 (Bartolome et al.; Gene 102, 75-78 (1991)), pTrc99A (Amann et al.; Gene 69, 301-315 (1988)) or pSC101 derivatives (Vocke and Bastia; Proceedings of the National Academy of Sciences USA 80(21), 6557-6561 (1983)), can be used. In one process according to the invention, it is possible to use a strain transformed with a plasmid vector, said plasmid vector carrying at least one or more genes selected from the group comprising lpd, aceE and aceF, or nucleotide sequences coding therefor.

Also, mutations which affect the expression of the appropriate genes can be transferred to different strains by sequence exchange (Hamilton et al.; Journal of Bacteriology 171, 4617-4622 (1989)), conjugation or transduction.

Furthermore, for the production of L-amino acids, especially L-threonine, with strains of the family Enterobacteriaceae, it can be advantageous not only to enhance one or more genes selected from the group comprising lpd, aceE and aceF, but also to enhance one or more enzymes of the known threonine biosynthetic pathway, or enzymes of the anaplerotic metabolism, or enzymes for the production of reduced nicotinamide adenine dinucleotide phosphate, or glycolytic enzymes, or PTS enzymes or enzymes of sulfur metabolism. The use of endogenous genes is generally preferred.

Thus, for example, one or more genes selected from the group comprising:
the thrABC operon coding for aspartate kinase, homoserine dehydrogenase, homoserine kinase and threonine synthase (U.S. Pat. No. 4,278,765),
the pyc gene coding for pyruvate carboxylase (DE-A-19 831 609),
the pps gene coding for phosphoenolpyruvate synthase (Molecular and General Genetics 231(2), 332-336 (1992)),
the ppc gene coding for phosphoenolpyruvate carboxylase (Gene 31, 279-283 (1984)),
the pntA and pntB genes coding for transhydrogenase (European Journal of Biochemistry 158, 647-653 (1986)),
the rhtB gene for homoserine resistance (EP-A-0 994 190),
the mqo gene coding for malate:quinone oxidoreductase (DE 100 348 33.5),
the rhtC gene for threonine resistance (EP-A-1 013 765),
the thrE gene of Corynebacterium glutamicum coding for threonine export protein (DE 100 264 94.8),
the gdhA gene coding for glutamate dehydrogenase (Nucleic Acids Research 11, 5257-5266 (1983); Gene 23, 199-209 (1983)),
the hns gene coding for DNA binding protein HLP-II (Molecular and General Genetics 212, 199-202 (1988)),
the pgm gene coding for phosphoglucomutase (Journal of Bacteriology 176, 5847-5851 (1994)),
the fba gene coding for fructose biphosphate aldolase (Biochemical Journal 257, 529-534 (1989)),
the ptsH gene of the ptsHIcrr operon coding for phosphohistidine protein hexose phosphotransferase of the phosphotransferase system PTS (Journal of Biological Chemistry 262, 16241-16253 (1987)),
the ptsI gene of the ptsHIcrr operon coding for enzyme I of the phosphotransferase system PTS (Journal of Biological Chemistry 262, 16241-16253 (1987)),
the crr gene of the ptsHIcrr operon coding for the glucose-specific IIA component of the phosphotransferase system PTS (Journal of Biological Chemistry 262, 16241-16253 (1987)),
the ptsG gene coding for the glucose-specific IIBC component (Journal of Biological Chemistry 261, 16398-16403 (1986)),
the lrp gene coding for the regulator of the leucine regulon (Journal of Biological Chemistry 266, 10768-10774 (1991)),
the csrA gene coding for the global regulator Csr (Journal of Bacteriology 175, 4744-4755 (1993)),
the fadR gene coding for the regulator of the fad regulon (Nucleic Acids Research 16, 7995-8009 (1988)),
the iclR gene coding for the regulator of the central intermediary metabolism (Journal of Bacteriology 172, 2642-2649 (1990)),
the mopB gene coding for the 10 kd chaperone (Journal of Biological Chemistry 261, 12414-12419 (1986)), which is also known as groES,
the ahpC gene of the ahpCF operon coding for the small subunit of alkyl hydroperoxide reductase (Proceedings of the National Academy of Sciences USA 92, 7617-7621 (1995)),
the ahpF gene of the ahpCF operon coding for the large subunit of alkyl hydroperoxide reductase (Proceedings of the National Academy of Sciences USA 92, 7617-7621 (1995)),
the cysK gene coding for cysteine synthase A (Journal of Bacteriology 170, 3150-3157 (1988)),
the cysB gene coding for the regulator of the cys regulon (Journal of Biological Chemistry 262, 5999-6005 (1987)),
the cysJ gene of the cysJIH operon coding for the flavoprotein of NADPH sulfite reductase (Journal of Biological Chemistry 264, 15796-15808 (1989), Journal of Biological Chemistry 264, 15726-15737 (1989)),
the cysI gene of the cysJIH operon coding for the hemoprotein of NADPH sulfite reductase (Journal of Biological Chemistry 264, 15796-15808 (1989), Journal of Biological Chemistry 264, 15726-15737 (1989)),
the cysH gene of the cysJIH operon coding for adenylyl sulfate reductase (Journal of Biological Chemistry 264, 15796-15808 (1989), Journal of Biological Chemistry 264, 15726-15737 (1989)),
the phoB gene of the phoBR operon coding for the PhoB positive regulator of the pho regulon (Journal of Molecular Biology 190 (1), 37-44 (1986)),
the phoR gene of the phoBR operon coding for the sensor protein of the pho regulon (Journal of Molecular Biology 192 (3), 549-556 (1986)),
the phoE gene coding for protein E of the outer cell membrane (Journal of Molecular Biology 163 (4), 513-532 (1983)),
the pykF gene coding for fructose-stimulated pyruvate kinase I (Journal of Bacteriology 177 (19), 5719-5722 (1995)),
the pfkB gene coding for 6-phosphofructokinase II (Gene 28 (3), 337-342 (1984)),
the malE gene coding for the periplasmatic binding protein of maltose transport (Journal of Biological Chemistry 259 (16), 10606-10613 (1984)),
the rseA gene of the rseABC operon coding for a membrane protein with anti-sigmaE activity (Molecular Microbiology 24 (2), 355-371 (1997)),
the rseC gene of the rseABC operon coding for a global regulator of the sigmaE factor (Molecular Microbiology 24 (2), 355-371 (1997)),
the soda gene coding for superoxide dismutase (Journal of Bacteriology 155 (3), 1078-1087 (1983)),
the sucA gene of the sucABCD operon coding for the decarboxylase subunit of 2-ketoglutarate dehydrogenase (European Journal of Biochemistry 141 (2), 351-359 (1984)),
the sucB gene of the sucABCD operon coding for the dihydrolipoyl transsuccinase E2 subunit of 2-ketoglutarate dehydrogenase (European Journal of Biochemistry 141 (2), 361-374 (1984)),
the sucC gene of the sucABCD operon coding for the β subunit of succinyl-CoA synthetase (Biochemistry 24 (22), 6245-6252 (1985)), and
the sucD gene of the sucABCD operon coding for the α subunit of succinyl-CoA synthetase (Biochemistry 24 (22), 6245-6252 (1985))
   can be simultaneously enhanced and, in particular, overexpressed.

Furthermore, for the production of L-amino acids, especially L-threonine, it can be advantageous not only to enhance one or more genes selected from the group comprising lpd, aceE and aceF, but also to attenuate and, in particular, switch off one or more genes selected from the group comprising:
the tdh gene coding for threonine dehydrogenase (Journal of Bacteriology 169, 4716-4721 (1987)),
the mdh gene coding for malate dehydrogenase (E.C. 1.1.1.37) (Archives in Microbiology 149, 36-42 (1987)),
the gene product of the open reading frame (orf) yjfA (Accession Number AAC77180 of the National Center for Biotechnology Information (NCBI, Bethesda, Md., USA)),
the gene product of the open reading frame (orf) ytfP (Accession Number AAC77179 of the National Center for Biotechnology Information (NCBI, Bethesda, Md., USA)),
the pckA gene coding for the enzyme phosphoenolpyruvate carboxykinase (Journal of Bacteriology 172, 7151-7156 (1990)),
the poxB gene coding for pyruvate oxidase (Nucleic Acids Research 14 (13), 5449-5460 (1986)),
the aceA gene coding for the enzyme isocitrate lyase (Journal of Bacteriology 170, 4528-4536 (1988)),
the dgsA gene coding for the DgsA regulator of the phosphotransferase system (Bioscience, Biotechnology and Biochemistry 59, 256-261 (1995)), which is also known as the mlc gene,
the fruR gene coding for the fructose repressor (Molecular and General Genetics 226, 332-336 (1991)), which is also known as the cra gene,
the rpoS gene coding for the sigma³⁸ factor (WO 01/05939), which is also known as the katF gene,
the aspA gene coding for aspartate ammonium lyase (aspartase) (Nucleic Acids Research 13 (6), 2063-2074 (1985)), and
the aceB gene coding for malate synthase A (Nucleic Acids Research 16 (19), 9342 (1988)),
   or reduce the expression.

In this context the term “attenuation” describes the decrease or switching-off of the intracellular activity, in a microorganism, of one or more enzymes (proteins) coded for by the appropriate DNA, for example by using a weak promoter or using a gene or allele which codes for an appropriate enzyme with a low activity or inactivates the appropriate enzyme (protein) or gene, and optionally combining these measures.

The attenuation measures generally reduce the activity or concentration of the appropriate protein to 0 to 75%, 0 to 50%, 0 to 25%, 0 to 10% or 0 to 5% of the activity or concentration of the wild-type protein or of the activity or concentration of the protein in the starting microorganism.

Furthermore, for the production of L-amino acids, especially L-threonine, it can be advantageous not only to enhance one or more genes selected from the group comprising lpd, aceE and aceF, but also to switch off unwanted secondary reactions (Nakayama: “Breeding of Amino Acid Producing Microorganisms”, in: Overproduction of Microbial Products, Krumphanzl, Sikyta, Vanek (eds.), Academic Press, London, UK, 1982).

The microorganisms prepared according to the invention can be cultivated by the batch process, the fed batch process or the repeated fed batch process. A summary of known cultivation methods is provided in the textbook by Chmiel (Bioprozesstechnik 1. Einführung in die Bioverfahrenstechnik (Bioprocess Technology 1. Introduction to Bioengineering) (Gustav Fischer Verlag, Stuttgart, 1991)) or in the textbook by Storhas (Bioreaktoren und periphere Einrichtungen (Bioreactors and Peripheral Equipment) (Vieweg Verlag, Brunswick/Wiesbaden, 1994)).

The culture medium to be used must appropriately meet the demands of the particular strains. Descriptions of culture media for various microorganisms can be found in “Manual of Methods for General Bacteriology” of the American Society for Bacteriology (Washington D.C., USA, 1981).

Carbon sources which can be used are sugars and carbohydrates, e.g. glucose, sucrose, lactose, fructose, maltose, molasses, starch and optionally cellulose, oils and fats, e.g. soya oil, sunflower oil, groundnut oil and coconut fat, fatty acids, e.g. palmitic acid, stearic acid and linoleic acid, alcohols, e.g. glycerol and ethanol, and organic acids, e.g. acetic acid. These substances can be used individually or as a mixture.

Nitrogen sources which can be used are organic nitrogen-containing compounds such as peptones, yeast extract, meat extract, malt extract, corn steep liquor, soya flour and urea, or inorganic compounds such as ammonium sulfate, ammonium chloride, ammonium phosphate, ammonium carbonate and ammonium nitrate. The nitrogen sources can be used individually or as a mixture.

Phosphorus sources which can be used are phosphoric acid, potassium dihydrogenphosphate or dipotassium hydrogenphosphate or the corresponding sodium salts. The culture medium must also contain metal salts, e.g. magnesium sulfate or iron sulfate, which are necessary for growth. Finally, essential growth-promoting substances such as amino acids and vitamins can be used in addition to the substances mentioned above. Suitable precursors can also be added to the culture medium. Said feed materials can be added to the culture all at once or fed in appropriately during cultivation.

The fermentation is generally carried out at a pH of 5.5 to 9.0, especially of 6.0 to 8.0. The pH of the culture is controlled by the appropriate use of basic compounds such as sodium hydroxide, potassium hydroxide, ammonia or aqueous ammonia, or acid compounds such as phosphoric acid or sulfuric acid. Foaming can be controlled using antifoams such as fatty acid polyglycol esters. The stability of plasmids can be maintained by adding suitable selectively acting substances, e.g. antibiotics, to the medium. Aerobic conditions are maintained by introducing oxygen or oxygen-containing gaseous mixtures, e.g. air, into the culture. The temperature of the culture is normally 25° C. to 45° C. and preferably 30° C. to 40° C. The culture is continued until the formation of L-amino acids or L-threonine has reached a maximum. This objective is normally achieved within 10 hours to 160 hours.

L-Amino acids can be analyzed by means of anion exchange chromatography followed by ninhydrin derivation, as described by Spackman et al. (Analytical Chemistry 30, 1190-1206 (1958)), or by reversed phase HPLC, as described by Lindroth et al. (Analytical Chemistry 51, 1167-1174 (1979)).

The process according to the invention is used for the preparation of L-amino acids, e.g. L-threonine, L-isoleucine, L-valine, L-methionine, L-homoserine and L-lysine, especially L-threonine, by fermentation.

The present invention is illustrated in greater detail below with the aid of Examples.

The minimum medium (M9) and complete medium (LB) used for Escherichia coli are described by J. H. Miller (A Short Course in Bacterial Genetics (1992), Cold Spring Harbor Laboratory Press). The isolation of plasmid DNA from Escherichia coli and all the techniques for restriction, ligation, Klenow treatment and alkaline phosphatase treatment are carried out according to Sambrook et al. (Molecular Cloning—A Laboratory Manual (1989), Cold Spring Harbor Laboratory Press). The transformation of Escherichia coli is carried out according to Chung et al. (Proceedings of the National Academy of Sciences of the United States of America 86, 2172-2175 (1989)) or according to Chuang et al. (Nucleic Acids Research 23, 1641 (1995)).

The incubation temperature in the preparation of strains and transformants is 37° C.

### EXAMPLE 1

Preparation of L-Threonine Using the lpd Gene

1a) Construction of Expression Plasmid pTrc99Alpd

The lpd gene from E. coli K12 is amplified using the polymerase chain reaction (PCR) and synthetic oligonucleotides. The nucleotide sequence of the lpd gene in E. coli K12 MG1655 (Accession Number AE000121, Blattner et al. (Science 277 (5331), 1453-1474 (1997))) is used as the starting material to synthesize PCR primers (MWG Biotech, Ebersberg, Germany). The 5′ ends of the primers are extended with recognition sequences for restriction enzymes and with two to four additional bases. This part of the primer is identified by a hyphen (−) in the representation below. The recognition sequences for NcoI and SalI, which are underlined in the nucleotide sequences shown below, are chosen for the 5′ and 3′ primers respectively:

(SEQ ID No. 1)

| | |
|---|---|
| lpd5: | 5′-CATGCCATGG-TGAAAGACGACGGGTATGAC-3′ |

(SEQ ID No. 2)

| | |
|---|---|
| lpd3: | 5′-ACGCGTCGAC-GGATGTTCCGGCAAACGAAA-3′ |

The chromosomal E. coli K12 MG1655 DNA used for the PCR is isolated with “Qiagen Genomic-tips 100/G” (QIAGEN, Hilden, Germany) in accordance with the manufacturer's instructions. An approx. 1500 bp DNA fragment can be amplified with the specific primers under standard PCR conditions (Innis et al. (1990) PCR Protocols. A Guide to Methods and Applications, Academic Press) using Pfu DNA polymerase (Promega Corporation, Madison, USA). The PCR product is ligated with vector pCR-Blunt II-TOPO (Zero Blunt TOPO PCR Cloning Kit, Invitrogen, Groningen, The Netherlands) in accordance with the manufacturer's instructions and transformed into the E. coli strain TOP10 (Invitrogen, Groningen, The Netherlands). Plasmid-carrying cells are selected on LB agar supplemented with 50 μg/ml of kanamycin. After isolation of the plasmid DNA, the vector is cleaved with the restriction enzymes NcoI and SalI and, after checking in 0.8% agarose gel, is called pCRBluntlpd. Vector pCRBluntlpd is then restricted with the restriction enzymes NcoI and SalI and, after separation in 0.8% agarose gel, the lpd fragment is isolated using the QIAquick Gel Extraction Kit (QIAGEN, Hilden, Germany). Vector pTrc99A (Pharmacia Biotech, Uppsala, Sweden) is cleaved with the enzymes NcoI and SalI and ligated with the isolated lpd fragment. The E. coli strain XL1-Blue MRF′ (Stratagene, La Jolla, USA) is transformed with the ligation mixture and plasmid-carrying cells are selected on LB agar supplemented with 50 μg/ml of ampicillin. The success of the cloning can be demonstrated, after isolation of the plasmid DNA, by control cleavage with the enzymes NcoI/SalI, EcoRV and DraIII. The plasmid is called pTrc99Alpd (FIG. 1).

1b) Preparation of L-Threonine with the Strain MG442/pTrc99Alpd

The L-threonine-producing E. coli strain MG442 is described in patent U.S. Pat. No. 4,278,765 and is deposited in the Russian National Collection for Industrial Microorganisms (VKPM, Moscow, Russia) as CMIM B-1628.

The strain MG442 is transformed with expression plasmid pTrc99Alpd, described in Example 1a, and with vector pTrc99A and plasmid-carrying cells are selected on LB agar supplemented with 50 μg/ml of ampicillin. This procedure yields the strains MG442/pTrc99lpd and MG442/pTrc99A. Chosen individual colonies are then multiplied further on minimum medium of the following composition: 3.5 g/l of Na₂HPO₄.2H₂O, 1.5 g/l of KH₂PO₄, 1 g/l of NH₄Cl, 0.1 g/l of MgSO₄.7H₂O, 2 g/l of glucose, 20 g/l of agar, 50 mg/l of ampicillin. The formation of L-threonine is verified in 10 ml batch cultures contained in 100 ml conical flasks. This is done by inoculating 10 ml of preculture medium of the following composition: 2 g/l of yeast extract, 10 g/l of (NH₄)₂SO₄, 1 g/l of KH₂PO₄, 0.5 g/l of MgSO₄.7H₂O, 15 g/l of CaCO₃, 20 g/l of glucose, 50 mg/l of ampicillin, and incubating for 16 hours at 37° C. and 180 rpm on an ESR incubator from Kuhner AG (Birsfelden, Switzerland). 250 μl of each of these precultures are transferred to 10 ml of production medium (25 g/l of (NH₄)₂SO₄, 2 g/l of KH₂PO₄, 1 g/l of MgSO₄.7H₂O, 0.03 g/l of FeSO₄.7H₂O, 0.018 g/l of MnSO₄-1H₂O, 30 g/l of CaCO₃, 20 g/l of glucose, 50 mg/l of ampicillin) and incubated for 48 hours at 37° C. The formation of L-threonine by the original strain MG442 is verified in the same way except that no ampicillin is added to the medium. After incubation the optical density (OD) of the culture suspension is determined using an LP2W photometer from Dr. Lange (Dusseldorf, Germany) at a measurement wavelength of 660 nm.

The concentration of L-threonine formed is then determined in the sterile-filtered culture supernatant using an amino acid analyzer from Eppendorf-BioTronik (Hamburg, Germany) by means of ion exchange chromatography and postcolumn reaction with ninhydrin detection.

Table 1 shows the result of the experiment.

TABLE 1

| | OD | L-threonine |
|---|---|---|
| Strain | (660 nm) | g/l |
| MG442 | 5.6 | 1.4 |
| MG442/pTrc99A | 3.8 | 1.3 |
| MG442/pTrc99Alpd | 4.3 | 2.4 |

### EXAMPLE 2

Preparation of L-Threonine Using the aceE and aceF Genes

2a) Construction of Expression Plasmid pTrc99AaceEF

The aceEF gene region from E. coli K12 is amplified using the polymerase chain reaction (PCR) and synthetic oligonucleotides. The nucleotide sequence of the aceE and aceF genes in E. coli K12 MG1655 (Accession Number AE000120, Blattner et al. (Science 277, 1453-1474 (1997)) is used as the starting material to synthesize PCR primers (MWG Biotech, Ebersberg, Germany). The sequence of a primer is modified to create a recognition site for a restriction enzyme. The recognition sequence for SacI, which is underlined in the nucleotide sequence shown below, is chosen for the aceEF1 primer:

| | |
|---|---|
| aceEF1: | |
| 5′-GATTGAGCTCTCCGGCGAGAGTTC-3′ | (SEQ ID No. 3) |
| aceEF2: | |
| 5′-ACCGGGTCGTTCTATCCGTC-3′ | (SEQ ID No. 4) |

The chromosomal E. coli K12 MG1655 DNA used for the PCR is isolated with “Qiagen Genomic-tips 100/G” (QIAGEN, Hilden, Germany) in accordance with the manufacturer's instructions. An approx. 4800 bp DNA fragment can be amplified with the specific primers under standard PCR conditions (Innis et al. (1990) PCR Protocols. A Guide to Methods and Applications, Academic Press) using Pfu DNA polymerase (Promega Corporation, Madison, USA). The PCR product is ligated with vector pCR-Blunt II-TOPO (Zero Blunt TOPO PCR Cloning Kit, Invitrogen, Groningen, The Netherlands) in accordance with the manufacturer's instructions and transformed into the E. coli strain TOP10F′. Plasmid-carrying cells are selected on LB agar supplemented with 50 μg/ml of kanamycin. After isolation of the plasmid DNA, the vector is cleaved with the restriction enzymes EcoRI and BstEII/XhoI and, after checking by separation in 0.8% agarose gel, is called pCRBluntaceEF.

Vector pCRBluntaceEF is then restricted with the restriction enzymes SacI and XbaI and, after separation in 0.8% agarose gel, the aceEF fragment is isolated using the QIAquick Gel Extraction Kit (QIAGEN, Hilden, Germany). Vector pTrc99A (Pharmacia Biotech, Uppsala, Sweden) is cleaved with the enzymes SacI and XbaI and ligated with the isolated aceEF fragment. The E. coli strain XL1-Blue MRF′ (Stratagene, La Jolla, USA) is transformed with the ligation mixture and plasmid-carrying cells are selected on LB agar supplemented with 50 μg/ml of ampicillin. The success of the cloning can be demonstrated, after isolation of the plasmid DNA, by control cleavage with the enzymes HindIII and PstI. The plasmid is called pTrc99AaceEF (FIG. 2).

2b) Preparation of L-Threonine with the Strain MG442/pTrc99AaceEF

The L-threonine-producing E. coli strain MG442 is described in patent U.S. Pat. No. 4,278,765 and is deposited in the Russian National Collection for Industrial Microorganisms (VKPM, Moscow, Russia) as CMIM B-1628.

The strain MG442 is transformed with expression plasmid pTrc99AaceEF, described in Example 2a, and with vector pTrc99A and plasmid-carrying cells are selected on LB agar supplemented with 50 μg/ml of ampicillin. This procedure yields the strains MG442/pTrc99aceEF and MG442/pTrc99A. Chosen individual colonies are then multiplied further on minimum medium of the following composition: 3.5 g/l of Na₂HPO₄.2H₂O, 1.5 g/l of KH₂PO₄, 1 g/l of NH₄Cl, 0.1 g/l of MgSO₄.7H₂O, 2 g/l of glucose, 20 g/l of agar, 50 mg/l of ampicillin. The formation of L-threonine is verified in 10 ml batch cultures contained in 100 ml conical flasks. This is done by inoculating 10 ml of preculture medium of the following composition: 2 g/l of yeast extract, 10 g/l of (NH₄)₂SO₄, 1 g/l of KH₂PO₄, 0.5 g/l of MgSO₄.7H₂O, 15 g/l of CaCO₃, 20 g/l of glucose, 50 mg/l of ampicillin, and incubating for 16 hours at 37° C. and 180 rpm on an ESR incubator from Kühner AG (Birsfelden, Switzerland). 250 μl of each of these precultures are transferred to 10 ml of production medium (25 g/l of (NH₄)₂SO₄, 2 g/l of KH₂PO₄, 1 g/l of MgSO₄.7H₂O, 0.03 g/l of FeSO₄.7H₂O, 0.018 g/l of MnSO₄.1H₂O, 30 g/l of CaCO₃, 20 g/l of glucose, 50 mg/l of ampicillin) and incubated for 48 hours at 37° C. The formation of L-threonine by the original strain MG442 is verified in the same way except that no ampicillin is added to the medium. After incubation the optical density (OD) of the culture suspension is determined using an LP2W photometer from Dr. Lange (Düsseldorf, Germany) at a measurement wavelength of 660 nm.

The concentration of L-threonine formed is then determined in the sterile-filtered culture supernatant using an amino acid analyzer from Eppendorf-BioTronik (Hamburg, Germany) by means of ion exchange chromatography and postcolumn reaction with ninhydrin detection.

Table 2 shows the result of the experiment.

TABLE 2

| | OD | L-threonine |
|---|---|---|
| Strain | (660 nm) | g/l |
| MG442 | 5.6 | 1.4 |
| MG442/pTrc99A | 3.8 | 1.3 |
| MG442/pTrc99AaceEF | 3.9 | 1.8 |

### BRIEF DESCRIPTION OF THE FIGURES

FIG. 1: Map of plasmid pTrc99Alpd containing the lpd gene

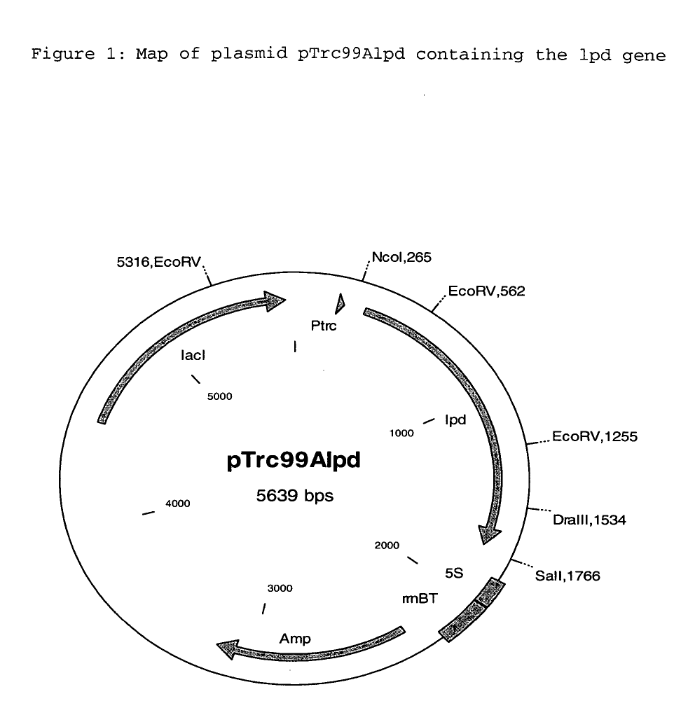

FIG. 2: Map of plasmid pTrac99AaceEF containing the aceE and aceF genes

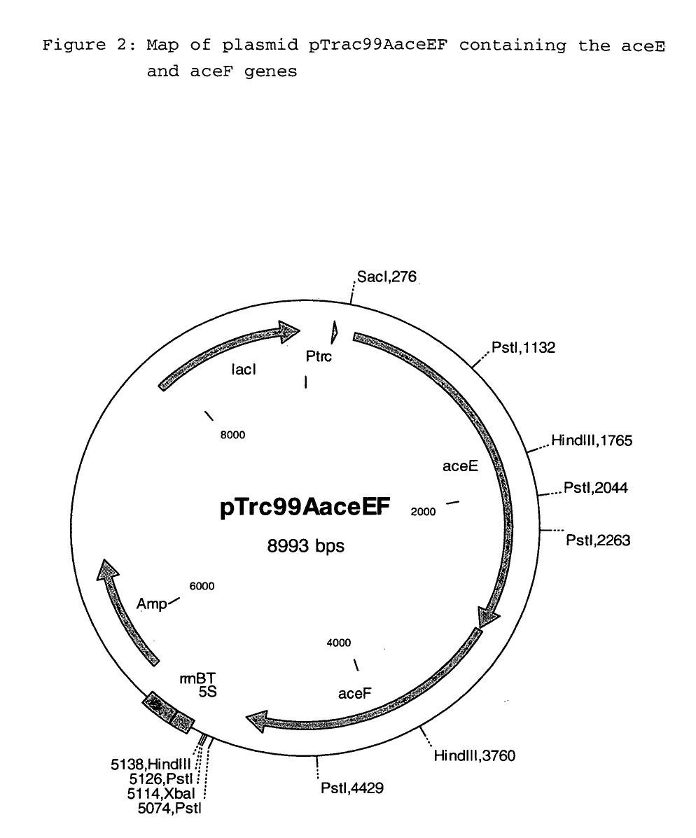

The indicated lengths are to be understood as approximate. The abbreviations and symbols used are defined as follows:
Amp: ampicillin resistance gene
lacI: gene for the repressor protein of the trc promoter
Ptrc: trc promoter region, IPTG-inducible
lpd: coding region of the lpd gene
aceE: coding region of the aceE gene
aceF: coding region of the aceF gene
5S: 5S rRNA region
rrnBT: rRNA terminator region
bps base pairs

The abbreviations for the restriction enzymes are defined as follows:
DraIII: restriction endonuclease from Deinococcus radiophilus
EcoRV: restriction endonuclease from Escherichia coli B946
HindIII: restriction endonuclease from Haemophilus influenzae
NcoI: restriction endonuclease from Nocardia corallina
PstI: restriction endonuclease from Providencia stuartii
SacI: restriction endonuclease from Streptomyces stanford
SalI: restriction endonuclease from Streptomyces albus
XbaI: restriction endonuclease from Xanthomonas badrii

1-8. (canceled)

9. A process for the preparation of an L-amino acid, comprising:
a) fermenting a modified microorganism of the family Enterobacteriaceae in a culture medium for a time and under conditions suitable for the production of the desired amino acid, wherein said modified microorganism overexpresses a polynucleotide encoding the product of a gene selected from the group consisting of: lpd; aceE; and aceF;
b) enriching said L-amino acid in said culture medium or in the microorganism fermented in step a); and
c) isolating said L-amino acid.

10. The process of claim 9, wherein constituents of the fermentation broth and/or all or part (>0 to 100%) of the biomass present after step b) remain in the product isolated in step c).

11. The process of claim 9, wherein said lpd; aceE; and aceF genes are obtainable from Enterobacteriaceae by PCR amplification using either:
a) primer lpd5 (SEQ ID NO:1) and primer lpd3 (SEQ ID NO:2) for the lpd gene; or
b) primer aceFE1 (SEQ ID NO:3) and primer aceFE2 (SEQ ID NO:4) for the aceE and aceF genes.

12. The process of claim 9, wherein said L-amino acid is selected from the group consisting of: L-threonine; L-serine; L-homoserine; L-valine; L-methionine; L-isoleucine; and L-lysine.

13. The process of claim 9, wherein said L-amino acid is L-threonine.

14. The process of claim 9, wherein at least one gene of the biosynthetic pathway of said L-amino acid is additionally enhanced in said microorganism.

15. The process of claim 9, wherein, in addition to the overexpression of a polynucleotide encoding the product of a gene selected from the group consisting of: lpd; aceE; and aceF, the expression of at least one gene of a metabolic pathway which reduces the amount of said L-amino acid in said microorganism is decreased or eliminated.

16. The process of claim 9, wherein the regulatory and/or catalytic properties of the polypeptide coded for by said polynucleotide are enhanced.

17. The process of claim 9, wherein said modified microorganism further comprises at least one overexpressed gene product compared to the unmodified microorganism, wherein said overexpressed gene product is encoded by a gene selected from the group consisting of:
a) at least one gene encoded by the thrABC operon which codes for aspartate kinase, homoserine dehydrogenase, homoserine kinase and threonine synthase;
b) a Corynebacterium glutamicum pyc gene coding for pyruvate carboxylase;
c) the pps gene coding for phosphoenolpyruvate synthase;
d) the ppc gene coding for phosphoenolpyruvate carboxylase;
e) the pntA and pntB genes coding for the subunits of pyridine transhydrogenase;
h) an Escherichia coli rhtC gene for a protein imparting threonine resistance;
i) a Corynebacterium glutamicum thrE gene coding for a threonine export carrier protein;
j) the gdhA gene coding for glutamate dehydrogenase;
k) the hns gene coding for DNA binding protein HLP-II;
l) the pgm gene coding for phosphoglucomutase;
m) the fba gene coding for fructose biphosphate aldolase;
n) the ptsH gene coding for phosphohistidine protein hexose phosphotransferase;
o) the ptsI gene coding for enzyme I of the phosphotransferase system;
p) the crr gene coding for the glucose-specific IIA component;
q) the ptsG gene coding for the glucose-specific IIBC component;
r) the Irp gene coding for the regulator of the leucine regulon;
s) the csrA gene coding for the global regulator Csr;
t) the fadR gene coding for the regulator of the fad regulon;
u) the ilcR gene coding for the regulator of central intermediary metabolism;
v) the mopB gene coding for the 10 kd chaperone;
w) the ahpC gene coding for the small subunit of alkyl hydroperoxide reductase;
x) the ahpF gene coding for the large subunit of alkyl hydroperoxide reductase;
y) the cysK gene coding for cysteine synthase A;
z) the cysB gene coding for the regulator of the cys regulon;
aa) the cysJ gene coding for the flavoprotein of NADPH sulfite reductase;
bb) the cysI gene coding for the hemoprotein of NADPH sulfite reductase;
cc) the cysH gene coding for adenylyl sulfate reductase;
dd) the phoB gene coding for the PhoB positive regulator of the pho regulon;
ee) the phoR gene coding for the sensor protein of the pho regulon;
ff) the phoE gene coding for protein E of the outer cell membrane;
gg) the pykF gene coding for fructose-stimulated pyruvate kinase I;
hh) the pfkB gene coding for 6-phosphofructokinase II;
ii) the malE gene coding for the periplasmatic binding protein of maltose transport;
jj) the rseA gene coding for a membrane protein with anti-sigmaE activity;
kk) the rseC gene coding for a global regulator of the sigmaE factor;
ll) the sodA gene coding for superoxide dismutase;
mm) the sucA gene coding for the decarboxylase subunit of 2-ketoglutarate dehydrogenase;
nn) the sucB gene coding for the dihydrolipoyl transsuccinase E2 subunit of 2-ketoglutarate dehydrogenase;
oo) the sucC gene coding for the β subunit of succinyl-CoA synthetase; and
pp) the sucD gene coding for the α subunit of succinyl-CoA synthetase.

18. The process of claim 9, wherein said modified microorganism further comprises at least one gene whose expression is reduced or eliminated compared to the unmodified microorganism, wherein said at least one gene is selected from the group consisting of:
a) the tdh gene coding for threonine dehydrogenase;
b) the mdh gene coding for malate dehydrogenase;
c) the gene product of the open reading frame (orf) yjfA of E. coli;
d) the gene product of the open reading frame (orf) ytfP of E. coli;
e) the pckA gene coding for phosphoenolpyruvate carboxykinase;
f) the poxB gene coding for pyruvate oxidase;
g) the aceA gene coding for isocitrate lyase;
h) the dgsA gene coding for the DgsA regulator of the phosphotransferase system;
i) the fruR gene coding for the fructose repressor;
j) the rpoS gene coding for the sigma³⁸ factor;
k) the aspA gene coding for aspartate ammonium lyase (aspartase); and
l) the aceB gene coding for malate synthase A.

19. A modified microorganism of the family Enterobacteriaceae in which a gene selected from the group consisting of: lpd; aceE; and aceF; or a polynucleotide coding for the gene product of said lpd, aceE and aceF gene is overexpressed.

20. The microorganism of claim 19, wherein said microorganism is of the genus Escherichia.

21. The microorganism of claim 20, wherein said microorganism is of the species Escherichia coli.

22. A process for the preparation of L-threonine, comprising:
a) fermenting an L-threonine-producing microorganism of the genus Escherichia in a culture medium, wherein said microorganism has been transformed with a vector comprising a polynucleotide encoding the product of a gene selected from the group consisting of: lpd; aceE; and aceF; said polynucleotide being obtainable from Escherichia by PCR amplification using either;
i) primer lpd5 (SEQ ID NO:1) and primer lpd3 (SEQ ID NO:2) for the lpd gene; or
ii) primer aceFE1 (SEQ ID NO:3) and primer aceF2 (SEQ ID NO:4) for the aceE and aceF genes; and
b) collecting L-threonine from either said culture medium or said microorganism after the fermentation of step a).

23. The process of claim 22, wherein said microorganism is of the species Escherichia coli.

24. The process of either claim 22 or claim 23, further comprising isolating said L-threonine from either said culture medium or said bacterium collected in step b).

25. The process of claim 24, wherein constituents of the fermentation broth and/or all or part (>0 to 100%) of the biomass remains present after isolating said L-threonine.

26. The process of claim 24, wherein said microorganism has been transformed with a polynucleotide comprising a promoter and encoding at least one additional gene of the biosynthetic pathway of L-threonine.

27. The process of claim 9, wherein said microorganism has been transformed with a polynucleotide comprising a promoter and encoding at least one additional gene selected from the group consisting of:
a) at least one gene encoded by the thrABC operon which codes for aspartate kinase, homoserine dehydrogenase, homoserine kinase and threonine synthase;
b) a Corynebacterium glutamicum pyc gene coding for pyruvate carboxylase;
c) the pps gene coding for phosphoenolpyruvate synthase;
d) the ppc gene coding for phosphoenolpyruvate carboxylase;
e) the pntA and pntB genes coding for the subunits of pyridine transhydrogenase;
h) an Escherichia coli rhtC gene for a protein imparting threonine resistance;
i) a Corynebacterium glutamicum thrE gene coding for a threonine export carrier protein;
j) the gdhA gene coding for glutamate dehydrogenase;
k) the hns gene coding for DNA binding protein HLP-II;
l) the pgm gene coding for phosphoglucomutase;
m) the fba gene coding for fructose biphosphate aldolase;
n) the ptsH gene coding for phosphohistidine protein hexose phosphotransferase;
o) the ptsI gene coding for enzyme I of the phosphotransferase system;
p) the crr gene coding for the glucose-specific IIA component;
q) the ptsG gene coding for the glucose-specific IIBC component;
r) the IrP gene coding for the regulator of the leucine regulon;
s) the csrA gene coding for the global regulator Csr;
t) the fadR gene coding for the regulator of the fad regulon;
u) the ilcR gene coding for the regulator of central intermediary metabolism;
v) the mopB gene coding for the 10 kd chaperone;
w) the ahpC gene coding for the small subunit of alkyl hydroperoxide reductase;
x) the ahpF gene coding for the large subunit of alkyl hydroperoxide reductase;
y) the cysK gene coding for cysteine synthase A;
z) the cysB gene coding for the regulator of the cys regulon;
aa) the cysJ gene coding for the flavoprotein of NADPH sulfite reductase;
bb) the cysI gene coding for the hemoprotein of NADPH sulfite reductase;
cc) the cysH gene coding for adenylyl sulfate reductase;
dd) the phoB gene coding for the PhoB positive regulator of the pho regulon;
ee) the phoR gene coding for the sensor protein of the pho regulon;
ff) the phoE gene coding for protein E of the outer cell membrane;
gg) the pykF gene coding for fructose-stimulated pyruvate kinase I;
hh) the pfkB gene coding for 6-phosphofructokinase II;
ii) the malE gene coding for the periplasmatic binding protein of maltose transport;
jj) the rseA gene coding for a membrane protein with anti-sigmaE activity;
kk) the rseC gene coding for a global regulator of the sigmaE factor;
ll) the sodA gene coding for superoxide dismutase;
mm) the sucA gene coding for the decarboxylase subunit of 2-ketoglutarate dehydrogenase;
nn) the sucB gene coding for the dihydrolipoyl transsuccinase E2 subunit of 2-ketoglutarate dehydrogenase;
oo) the sucC gene coding for the β subunit of succinyl-CoA synthetase; and
pp) the sucD gene coding for the α subunit of succinyl-CoA synthetase.

28. A microorganism of the genus Escherichia wherein said microorganism has been transformed with a polynucleotide comprising a promoter and encoding the protein of a gene selected from the group consisting of: lpd (NCBI accession number AE000121); aceE (NCBI accession number AE000120); and aceF (NCBI accession number AE000120).

29. The microorganism of claim 28, wherein said microorganism is of the species Escherichia coli.

30. The microorganism of either claim 28 or claim 29, wherein said microorganism also been transformed with a polynucleotide comprising a promoter and encoding at least one gene selected from the group consisting of:
a) at least one gene encoded by the thrABC operon which codes for aspartate kinase, homoserine dehydrogenase, homoserine kinase and threonine synthase;
b) a Corynebacterium glutamicum pyc gene coding for pyruvate carboxylase;
c) the pps gene coding for phosphoenolpyruvate synthase;
d) the ppc gene coding for phosphoenolpyruvate carboxylase;
e) the pntA and pntB genes coding for the subunits of pyridine transhydrogenase;
h) an Escherichia coli rhtC gene for a protein imparting threonine resistance;
i) a Corynebacterium glutamicum thrE gene coding for a threonine export carrier protein;
j) the gdhA gene coding for glutamate dehydrogenase;
k) the hns gene coding for DNA binding protein HLP-II;
l) the pgm gene coding for phosphoglucomutase;
m) the fba gene coding for fructose biphosphate aldolase;
n) the ptsH gene coding for phosphohistidine protein hexose phosphotransferase;
o) the ptsI gene coding for enzyme I of the phosphotransferase system;
p) the crr gene coding for the glucose-specific IIA component;
q) the ptsG gene coding for the glucose-specific IIBC component;
r) the Irp gene coding for the regulator of the leucine regulon;
s) the csrA gene coding for the global regulator Csr;
t) the fadR gene coding for the regulator of the fad regulon;
u) the ilcR gene coding for the regulator of central intermediary metabolism;
v) the mopB gene coding for the 10 kd chaperone;
w) the ahpC gene coding for the small subunit of alkyl hydroperoxide reductase;
x) the ahpF gene coding for the large subunit of alkyl hydroperoxide reductase;
y) the cysK gene coding for cysteine synthase A;
z) the cysB gene coding for the regulator of the cys regulon;
aa) the cysJ gene coding for the flavoprotein of NADPH sulfite reductase;
bb) the cysI gene coding for the hemoprotein of NADPH sulfite reductase;
cc) the cysH gene coding for adenylyl sulfate reductase;
dd) the phoB gene coding for the PhoB positive regulator of the pho regulon;
ee) the phoR gene coding for the sensor protein of the pho regulon;
ff) the phoE gene coding for protein E of the outer cell membrane;
gg) the pykF gene coding for fructose-stimulated pyruvate kinase I;
hh) the pfkB gene coding for 6-phosphofructokinase II;
ii) the malE gene coding for the periplasmatic binding protein of maltose transport;
jj) the rseA gene coding for a membrane protein with anti-sigmaE activity;
kk) the rseC gene coding for a global regulator of the sigmaE factor;
ll) the sodA gene coding for superoxide dismutase;
mm) the sucA gene coding for the decarboxylase subunit of 2-ketoglutarate dehydrogenase;
nn) the sucB gene coding for the dihydrolipoyl transsuccinase E2 subunit of 2-ketoglutarate dehydrogenase;
oo) the sucC gene coding for the β subunit of succinyl-CoA synthetase; and
pp) the sucD gene coding for the α subunit of succinyl-CoA synthetase.
